(19) United States
(12) Patent Application Publication
(10) Pub. No.: US 2010/0056912 A1
(43) Pub. Date: Mar. 4, 2010

(54) METHOD AND APPARATUS FOR AUTOMATICALLY DOWNLOADING MEDICAL IMAGING DATA

(75) Inventors: Mark Steven Urness, Wauwatosa, WI (US); Andrew Stonefield, Whitefish Bay, WI (US)

Correspondence Address: DEAN D. SMALL;THE SMALL PATENT LAW GROUP LLP, 225 S. MERAMEC, STE. 725T, ST. LOUIS, MO, 63105, US

(73) Assignee: General Electric Company, Schenectady, NY (US)

(21) Appl. No.: 12/199,501

(22) Filed: Aug. 27, 2008

(51) Int. Cl. A61B 8/00; G06F 17/30

(52) U.S. Cl. 600/437; 707/1; 707/E17.005

(57) ABSTRACT

A portable medical imaging device includes a user interface configured to receive a user input and a display area on the user interface configured to display medical images. The device also includes a processor module coupled to the user interface, the processor module configured to generate medical information, and a transmitter/receiver configured to receive a wireless signal from a remote medical system and automatically transmit the medical information to the remote medical system in response to the received wireless signal. A medical imaging system including the portable medical imaging device and a method for operating the medical imaging system are also provided.

### BACKGROUND OF THE INVENTION

This invention relates generally to medical imaging devices, and more particularly to a method and apparatus for acquiring and automatically downloading medical imaging data.

Many hospitals have ultrasound imaging devices that are used in a wide variety of medical imaging applications. The type of ultrasound devices used in hospitals typically include a computer, a monitor, a beamformer, and a keyboard that are mounted either permanently or on a mobile cart that may be moved from location to location to image patients within the hospital. With recent advances in imaging technology many portable or hand carried ultrasound devices are available that are considerably lighter than the types of ultrasound devices used by hospitals. The portable ultrasound imaging devices allow an operator to perform medical imaging on patients that are not located at the hospital. For example, portable ultrasound imaging devices may be used at nursing homes, in ambulances, or at clinics. The portable ultrasound systems typically include an ultrasound probe, a computer, and a keyboard. For example, the portable ultrasound device may be embodied as a laptop computer having a computer portion including a mechanical keyboard and a screen that is movable with respect to the keyboard. Optionally the portable ultrasound devices may be embodied as a single handheld unit including the computer, the screen, and the mechanical keyboard.

During operation, a operator typically receives the portable ultrasound device at a central location such as a hospital. The operator then uses the portable ultrasound device to acquire images of patients that are located outside of the hospital setting. For example, the portable ultrasound device may be used by ambulance personnel to scan patients at their homes or in transit to the hospital. The patient scans or patient data is then stored on a hard drive in the ultrasound device. Because, the storage capacity of the portable ultrasound device hard drive is limited, the operator is required to frequently download the patient data to free up space on the hard drive. Typically, the patient data is downloaded to a computer that is located at a central facility such as the hospital. To download the ultrasound data, the operator physically connects the portable device to the hospital network which includes the central computer. The portable device may also be connected through a docking station that has a physical connection to the hospital network. The operator then enters any patient information that is required by the hospital to associate the data with the patient for which the data was acquired.

The time required by the operator to download patient data at the hospital reduces the amount of time the operator may spend performing other duties such as performing scans. To reduce the frequency of required downloads, the ultrasound device may be retrofitted to include a hard drive having an increased capacity. However, increasing the capacity of the hard drive also results in a comparable increase in the cost of the ultrasound device and the weight of the ultrasound device. Optionally additional ultrasound devices may be purchased such that the operator receives and uses a second ultrasound device while the first ultrasound device is downloading data. However, increasing the quantity of ultrasound devices also increases the operational costs of the hospital and thus patient care costs in general.

### BRIEF DESCRIPTION OF THE INVENTION

In one embodiment, a portable medical imaging device is provided. The device includes a user interface configured to receive a user input and a display area on the user interface configured to display medical images. The device also includes a processor module coupled to the user interface, the processor module configured to generate medical information, and a transmitter/receiver configured to receive a wireless signal from a remote medical system and automatically transmit the medical information to the remote medical system in response to the received wireless signal.

In another embodiment, a medical imaging system is provided. The imaging system includes a data storage device, a transmitter/receiver coupled to the data storage device, and a portable medical imaging device having medical information stored thereon. The portable medical imagine device is configured to receive a wireless signal from transmitter/receiver and automatically transmit the medical information to the data storage device in response to the received signal.

In a further embodiment a method for downloading medical information is provided. The method includes generating the medical information using a portable medical imaging device and storing the medical information directly to a removable memory device installed in a portable medical imaging device. The method also includes receiving at a portable medical imaging device a radio-frequency (RF) signal and downloading medical information from the portable medical imaging device to a remote medical system in response to the received RF signal.

### BRIEF DESCRIPTION OF THE DRAWINGS

FIG. 1 is a block diagram of an exemplary medical imaging system.

FIG. 2 is a block diagram of the exemplary ultrasound scanner shown in FIG. 1.

FIG. 3 is a block diagram of the exemplary ultrasound processor module shown in FIG. 2.

FIG. 4 is a perspective view of the exemplary ultrasound scanner shown in FIGS. 1-3.

FIG. 5 is a front view of an exemplary display that may be viewed on the ultrasound scanner shown in FIG. 4.

FIG. 6 is a flowchart illustrating an exemplary method for downloading data.

### DETAILED DESCRIPTION OF THE INVENTION

The foregoing summary as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory hard disk, or the like). Similarly, the programs may be stand alone programs, may be incorporated as subroutines in an operating system may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

It should be noted that although the various embodiments may be described in connection with an ultrasound system the methods and systems described herein are not limited to ultrasound imaging. In particular, the various embodiments may be implemented in connection with different types of medical imaging, including, for example, magnetic resonance imaging (MRI) and computed-tomography (CT) imaging. Further, the various embodiments may be implemented in other non-medical imaging systems, for example, non-destructive testing systems.

Exemplary embodiments of ultrasound systems and methods for downloading information are described in detail below. In particular, a detailed description of an exemplary ultrasound system will first be provided followed by a detailed description of various embodiments of methods and systems for automatically downloading patient information to a central computer.

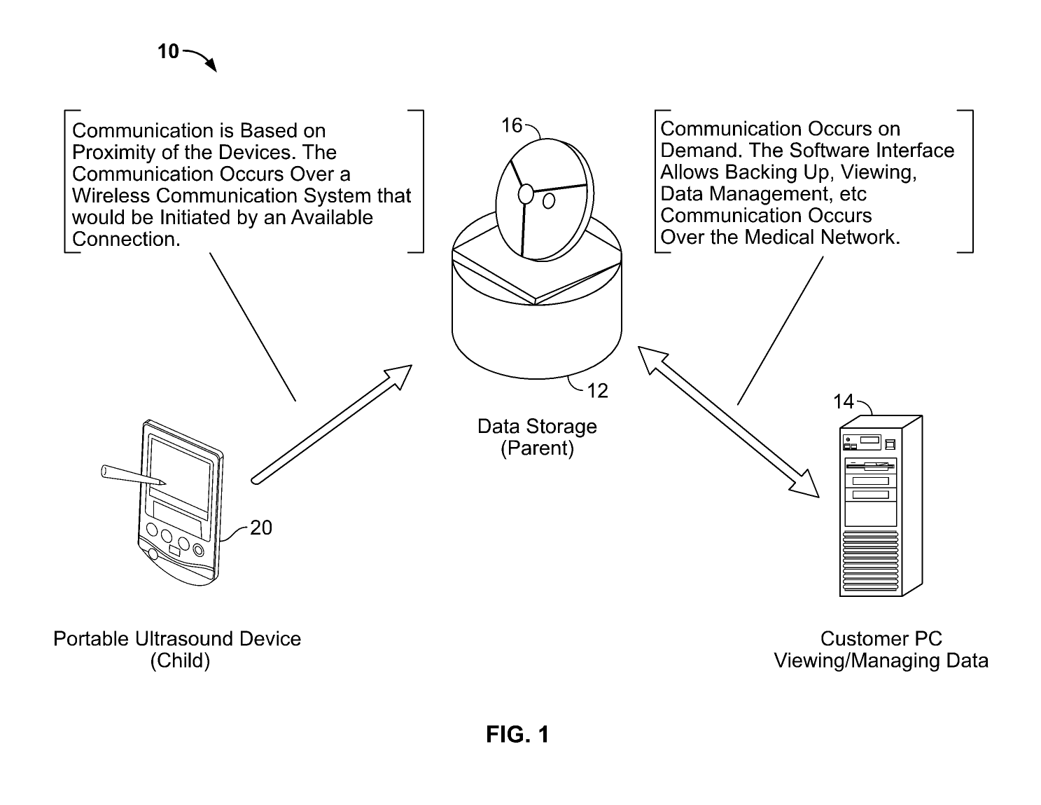

FIG. 1 illustrates a block diagram of an exemplary medical imaging system 10. In the exemplary embodiment the medical imaging system 10 is an ultrasound imaging system. The ultrasound imaging system 10 includes a data storage device 12, a computer 14, and an ultrasound scanner 20. In the exemplary embodiment, the data storage device 12 is embodied as a computer that includes a wireless transmitter/receiver 16 that is configured to transmit signals to the ultrasound scanner 20 and receive signals from the ultrasound scanner 20. In the exemplary embodiment, the data storage device 12 is located at a central facility such as a hospital and the ultrasound scanner 20 is a portable ultrasound scanner that is used at locations remote from the data storage device 12 as is discussed in more detail below. Although the imaging system 10 is described herein with respect to an ultrasound imaging system, it should be realized that the ultrasound imaging system is exemplary and that the various features described herein may be implemented on other portable imaging systems or non-medical imaging systems.

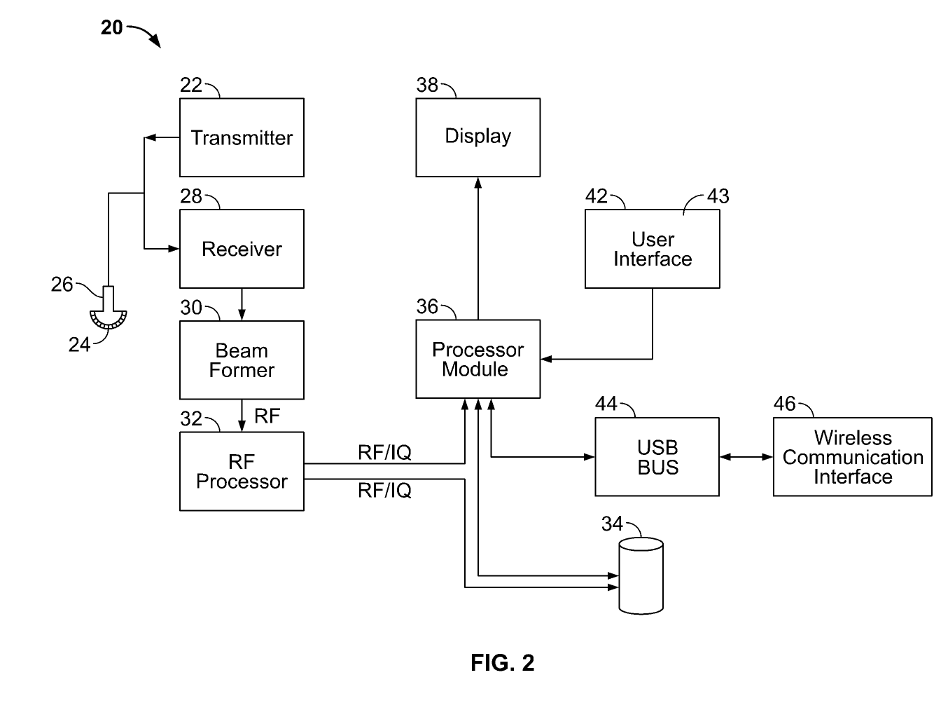

FIG. 2 is a block diagram of the ultrasound scanner 20 shown in FIG. 1. In the exemplary embodiment, the ultrasound scanner 20 includes a transmitter 22 that drives an array of elements 24 (e.g., piezoelectric crystals) within a transducer 26 to emit pulsed ultrasonic signals into a body or volume. A variety of geometries may be used and the transducer 26 may be provided as part of, for example, different types of ultrasound probes. The ultrasonic signals are back-scattered from structures in the body for example, blood cells or muscular tissue, to produce echoes that return to the elements 24. The echoes are received by a receiver 28. The received echoes are provided to a beamformer 30 that performs beamforming and outputs an RF signal. The RF signal is then provided to an RF processor 32 that processes the RF signal. Alternatively, the RF processor 32 may include a complex demodulator (not shown) that demodulates the RF signal to form IQ data pairs representative of the echo signals. In the exemplary embodiment, the ultrasound scanner 20 does not include a hard drive or other memory storage device that is configured to be permanently installed within the ultrasound scanner 20. As such, in one embodiment the RF or IQ signal data may be provided directly to a removable memory device 34 for storage (e.g., temporary storage). In the exemplary embodiment the removable memory device 34 may be implemented using a removable flash-based random access memory (RAM) device. Optionally the RF or IQ signal data may be provided to a processor module 36 to process the acquired ultrasound information (e.g., RF signal data or IQ data pairs) and prepare frames of ultrasound information for display on a display area 38 located on the user interface 42. The processor module 36 may then be programmed to download either the racy image data or the ultrasound images onto the removable memory device 34. In another embodiment, the ultrasound scanner includes a hard drive (not shown).

The processor module 36 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the memory device 34 during a scanning session and processed in less than real-time in a live or off-line operation. More specifically, the removable memory device 34 may be used for storing processed frames of acquired ultrasound information that are not scheduled to be displayed immediately. The processor module 36 is connected to a user interface 42 that controls some operations of the processor module 36 as explained below in more detail and is configured to receive inputs from an operator.

In the exemplary embodiment, the user interface 42 is a touch screen 43 and the display area 38 is programmed to display information on the touch screen 43. The touch screen 43 is also configured to detect the presence and location of a touch on the touch screen. The touch screen 43 may detect the presence of a finger or hand or the presence of a mechanical device such as a stylus. In the exemplary embodiment, the touch screen 43 performs the functions of a trackball, function keys, and the like. The touch screen 43 may be implemented as a resistive, capacitive, or other touch screen that provides an indication to the processor module 36 that an operator has touched the touch screen 43 and a location of the touch.

The display areas 38 includes one or more monitors that present patient information, including diagnostic ultrasound images to the user for review, diagnosis and analysis. The display area 38 may automatically display, for example, planes from two-dimensional (2D) and/or three-dimensional (3D) ultrasound data sets stored in the memory device 34. The memory device 34 may store 3D data sets of the ultrasound data, where such 3D data sets are accessed to present 2D and 3D images. The processing of the data, including the data sets, is based in part on user inputs, for example, user selections received at the touch screen 43. In the exemplary embodiment, the ultrasound scanner 20 also includes a transmitter/receiver, e.g. a wireless universal service bus (USB) 44 and a wireless communication interface 46 that is configured to transmit information from either the processor module 36 or the removable memory device 34 to the data storage device 12 (shown in FIG. 1) via the USB 44. In the exemplary embodiment, the wireless communication interface 46 may be implemented using a wireless USB device, a wireless Ethernet device, or a wireless broadband device.

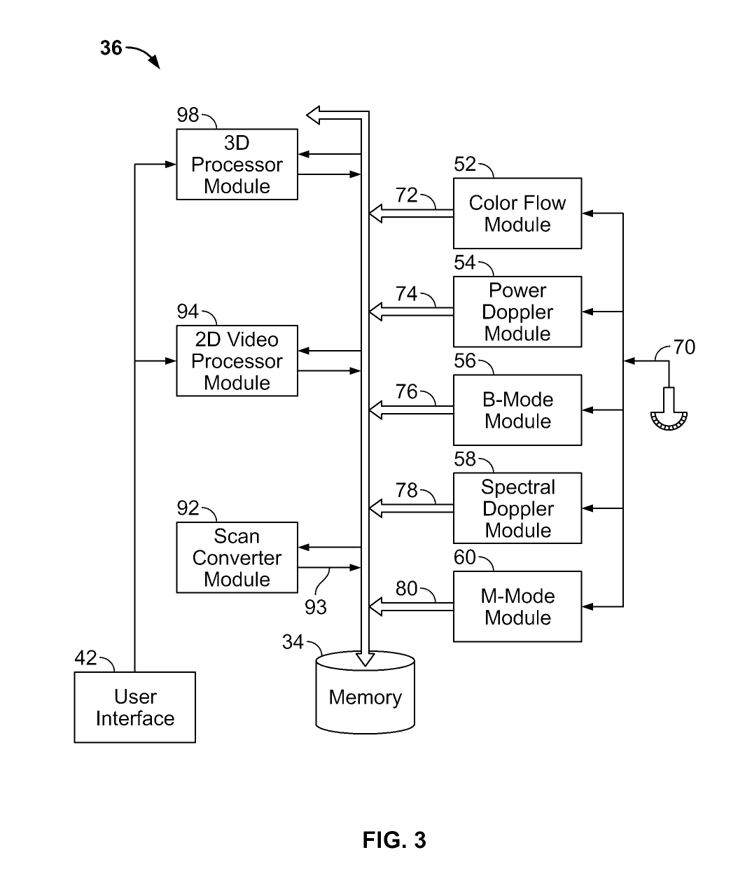

FIG. 3 illustrates an exemplary block diagram of the ultrasound processor module 36 of FIG. 2. The ultrasound processor module 36 is illustrated conceptually as a collection of sub-modules, but may be implemented utilizing any combination of dedicated hardware boards, DSPs, processors, etc. Alternatively, the sub-modules of FIG. 3 may be implemented utilizing an off-the-shelf PC with a single processor or multiple processors, with the functional operations distributed between the processors. As a further option, the sub-modules of FIG. 3 may be implemented utilizing a hybrid configuration in which certain modular functions are performed utilizing dedicated hardware, while the remaining modular functions are performed utilizing an off-the-shelf PC and the like. The sub-modules also may be implemented as software modules within a processing unit.

In the exemplary embodiment, the operations of the sub-modules illustrated in FIG. 3 are controlled by the touch screen 43 or by the processor module 36. The sub-modules 52-60 perform mid-processor operations. The ultrasound processor module 36 may receive ultrasound data 70 in one of several forms. In the embodiment of FIG. 3, the received ultrasound data 70 constitutes IQ data pairs representing the real and imaginary components associated with each data sample. The IQ data pairs are provided to one or more of a color-flow sub-module 52, a power Doppler sub-module 54, a B-mode sub-module 56, a spectral Doppler sub-module 58 and an M-mode sub-module 60.

Each of sub-modules 52-60 are configured to process the IQ data pairs in a corresponding manner to generate color-flow data 72, power Doppler data 74, B-mode data 76, spectral Doppler data 78, and M-mode data 80, all of which may be stored in the memory device 34 temporarily before subsequent processing. The data 72-80 may be stored, for example, as sets of vector data values, where each set defines an individual ultrasound image frame. The vector data values are generally organized based on the polar coordinate system.

A scan converter sub-module 92 accesses and obtains from the memory device 34 the vector data values associated with an image frame and converts the set of vector data values to Cartesian coordinates to generate an ultrasound image frame 93 formatted for display. The ultrasound image frames 93 generated by the scan converter sub-module 92 may be provided back to the memory device 34 for subsequent processing.

A 2D video processor sub-module 94 may be used to combine one or more of the frames generated from the different types of ultrasound information. For example, the 2D video processor sub-module 94 may combine different image frames by mapping one type of data to a gray map and mapping the other type of data to a color map for video display. In the final displayed image, the color pixel data is superimposed on the gray scale pixel data to form a single multi-mode image frame that is again re-stored in the memory device 34. Successive frames of images may be stored as a cine loop in the memory device 34. The cine loop represents a first in, first out circular image buffer to capture image data that is displayed in real-time to the user, such as one or more heart cycles. The user may freeze the cine loop by entering a freeze command at the touch screen.

A 3D processor sub-module 98 is also controlled by the touch screen 43 and accesses the memory device 34 to obtain spatially consecutive groups of ultrasound image frames and to generate three dimensional image representations thereof, such as through volume rendering or surface rendering algorithms as are known. The three dimensional images may be generated utilizing various imaging techniques, such as ray-casting, maximum intensity pixel projection and the like. In operation, the ultrasound scanner 20 acquires data, for example, volumetric data sets by various techniques (e.g., 3D scanning, real-time 3D imaging, volume scanning, 2D scanning with transducers having positioning sensors, freehand scanning using a voxel correlation technique, scanning using 2D or matrix array transducers, etc.).

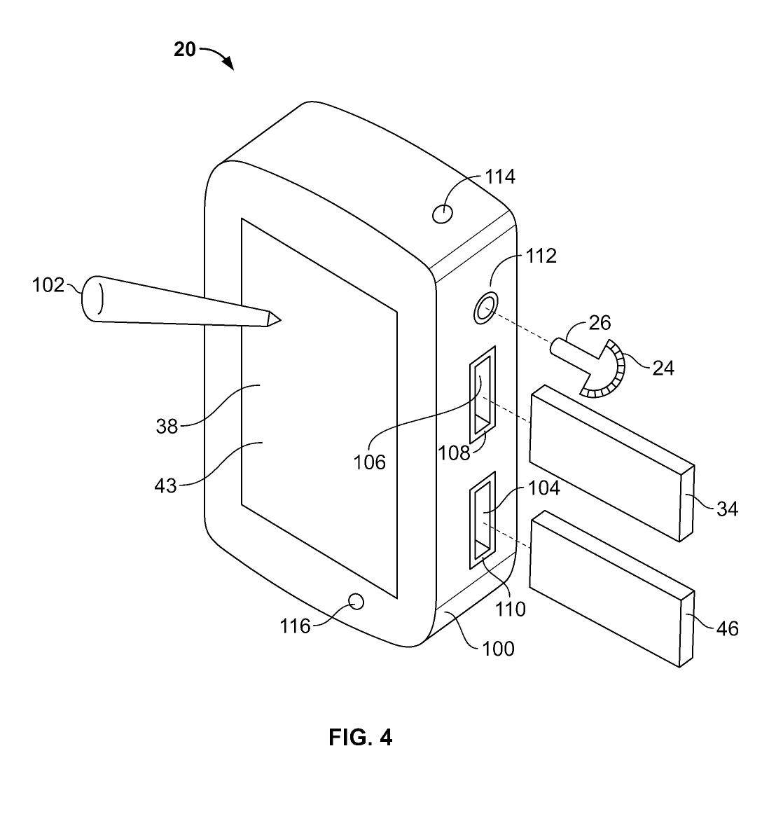

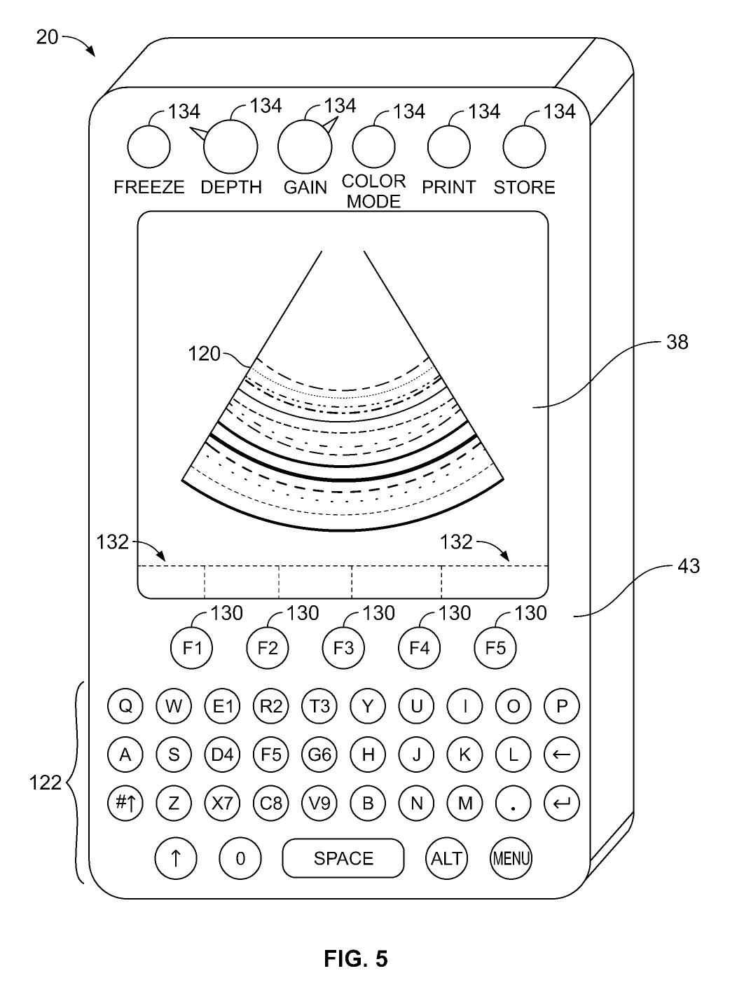

FIG. 4 is a perspective view of the ultrasound scanner 20 shown in FIGS. 1-3. FIG. 5 is a front view of an exemplary display that may be observed using the ultrasound scanner 20 shown in FIG. 4. In the exemplary embodiment, the ultrasound scanner 20 is a hand carried or portable ultrasound imaging system. By way of example, the ultrasound scanner may be a pocket-sized, hand-sized or laptop sized ultrasound system that is approximately 8.5 inches wide and approximately 11 inches in length. The ultrasound scanner 20 includes a body 100, the touch screen 43, and the display area 38 programmed on the touch screen 43. The touch screen 43 may be used to operate various features or controls shown on the display area 38. In the one embodiment, the touch screen 43 is operable using a stylus 102. Optionally the touch screen 43 may be operated by an operator using, for example, their finger.

The ultrasound scanner 20 also includes a first port 104 that is configured to receive the wireless communication interface 46 and a second port 106 that is configured to receive the removable memory device 34. Each of the first and second ports includes a gasket or seal 108 and 110, respectively. The gasket 108 is configured to form a seal between the port 106 and the removable memory device 34 when the removable memory device 34 is inserted into the port 106. The gasket 1 10 is configured to form a seal between the port 104 and the wireless communication interface 46 when the wireless communication interface 46 is inserted into the port 104. The gaskets or seals 108 and 110 substantially prevent dirt, moisture or other foreign substances from entering the body 100. More specifically in the exemplary embodiment, the ultrasound scanner 20 is substantially sealed during fabrication to reduce or eliminate possible contaminants from entering the unit and to improve the operator's ability to easily clean the ultrasound scanner 20. For example, during typical use, the ultrasound scanner may be subjected to bodily fluids. To clean known ultrasound scanners having mechanically operated keyboards, the operator typically expends a significant amount of time cleaning or sterilizing the unit prior to each use. In this case, because the ultrasound scanner 20 includes a touch screen 43 that is hermetically sealed to the body 100, the time required to clean the ultrasound scanner 20 between uses is substantially reduced. The ultrasound scanner 20 also includes a port 112 configured to receive the ultrasound probe 26, a port 114 configured to receive a power input for charging or powering the ultrasound scanner 20, and a power on/off button 116. In the exemplary embodiment the port 114 may be configured to mate with a respective port on a suitable charging station.

FIG. 5 is a front view of the exemplary display area 38 that may be viewed on the ultrasound scanner 20 shown in FIG. 4. It should be realized that the display area 38 may be configured, using the touch screen 43, to display any of the functions described herein and the functions illustrated in FIG. 5 are only exemplary. The display area 38 may be, for example, an 8.5 inch by 11 inch color LCD display (on which a medical image 120 may be displayed). A keyboard of buttons 122 may optionally be shown on the display area 38 and operable using the touch screen 43.

The ultrasound scanner 20 may also include multi-function controls 130 which may each be assigned functions in accordance with the mode of system operation. Therefore, each of the multi-function controls 130 may be configured to provide a plurality of different actions. Label display areas 132 associated with the multi-function controls 130 may be included as necessary on the display area 38. The ultrasound scanner 20 may also have additional keys and/or controls 134 for special purpose functions, which may include, but are not limited to “freeze,” “depth control,” “gain control,” “color-mode,” “print,” and “store.” As discussed above, all of the controls and functions described herein are operable using the touch screen 43.

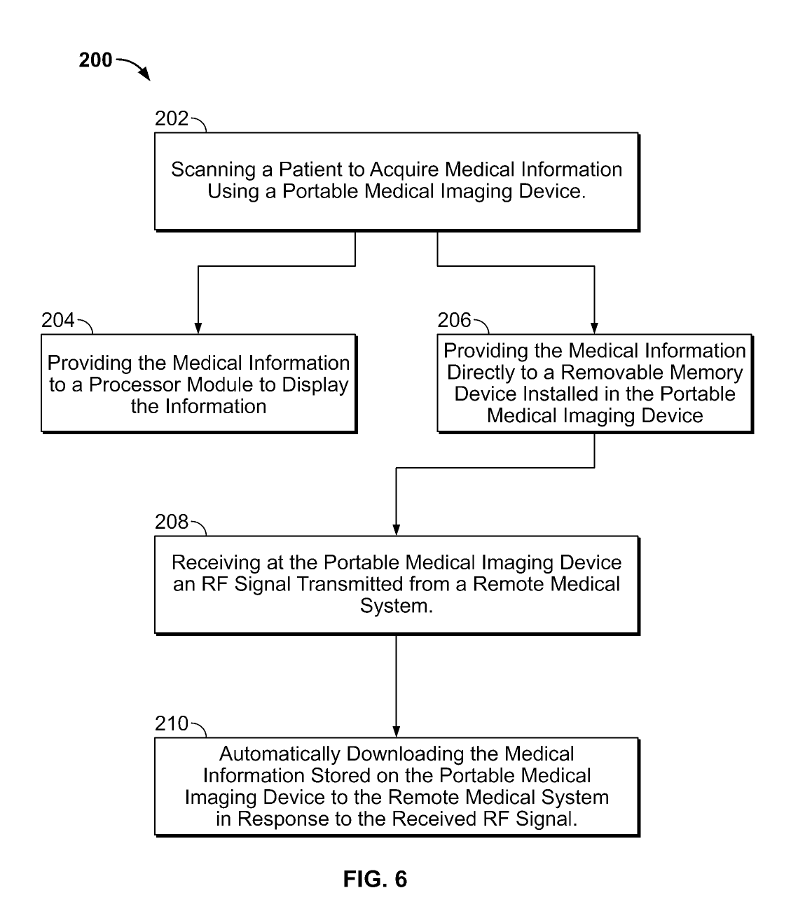

FIG. 6 is a flowchart illustrating an exemplary method 200 for downloading medical imaging information or data. At step 202 the patient or object is scanned to acquire information. In the exemplary embodiment a patient is scanned to acquire ultrasound medical information of a patient. At step 204, the patient information is provided to the processor module 36 to process the acquired ultrasound information (e.g., RF signal data or IQ data pairs) and prepare frames of ultrasound information for display on a display area 38. Optionally at step 206, the patient information is provided directly to a removable memory device 34 for storage (e.g., temporary storage). It should be realized that the quantity of medical information that may be stored on the removable memory device 34 is based on the memory storage capability of the memory storage device itself.

During typical operation, the operator may scan one or multiple patients to acquire medical information. After the patient scanning is completed, the operator marks the patient data to assign the patient data to the patient from which that data was acquired. For example, the operator may use the keys 122, via the touch screen 43, to type in the patients name and any or identifying information. This identifying information is then used to identify or label the data files that represent the scanned information. As discussed above, because the size of the removable memory device 34 is limited, the operator is required to frequently download the patient data. As discussed above, during typical operation the ultrasound scanner 20 is used remotely from the central location, e.g., the hospital. When the operator returns to the hospital, the patient data is downloaded to the central computer, e.g., data storage device 12. To facilitate automatic data download from the ultrasound scanner 20 to the data storage device 12, the wireless transmitter/receiver 16 is activated to output an RF signal that is to be received by the ultrasound scanner 20. In one embodiment, the RF signal is continuously output from the wireless transmitter/receiver 16 at a predetermined frequency and the ultrasound scanner 20 is configured to monitor the predetermined frequency. For example, assuming that the system 10 includes a plurality of ultrasound scanners 20, each ultrasound scanner may be configured to operate at a different frequency and the wireless transmitter/receiver 16 may be configured to transmit different RF signals at a frequency that corresponds to the operational frequencies of the ultrasound scanners 20 in operation. Optionally the plurality of ultrasound scanners 20 may be configured to operate at the same frequency. Optionally the RF signal is output at intervals that are sufficient to enable the medical information to be automatically downloaded from the ultrasound scanner 20.

Accordingly, at step 208 the ultrasound scanner 20 receives the wireless RF signal from the wireless transmitter/receiver 16. In the exemplary embodiment, the ultrasound scanner 20 utilizes the wireless communication interface 46 to receive the signal transmitted from the wireless transmitter/receiver 16 which functions as a wireless universal service bus (WUSB) hub. Wireless USB, as used herein is a relatively short-range a short-range, high-bandwidth wireless radio communication protocol that is capable of transmitting and receiving, for example, 480 M/bits at distances up to approximately 3 meters and 110 Mbit/s at distances up to approximately 1I meters. For example, when the operator carrying the ultrasound scanner 20 passes within a predetermined distance, e.g. less than 10 meters, from the wireless transmitter/receiver 16, the wireless communication interface 46 of the ultrasound scanner receives the RF signal being transmitted. In response to the received signal, at step 210 the ultrasound scanner 20 automatically downloads the information stored on the removable memory device 34 through the USB hub 44 to the data storage device 12 via the wireless communication interface 46. In another exemplary embodiment, the wireless universal service bus (WUSB) hub, e.g. transmitter/receiver 16, may be located at any convenient location and the patient information may be downloaded from the transmitter/receiver 16 to the data storage device 12 using the Internet or a modem, for example.

Described herein is a method and device to facilitate electronic data delivery. The device is embodied as a portable hand carried ultrasound imaging device that is programmed to discover or identify another mobile or permanently installed device within its vicinity and wirelessly download medical data to the device when prompted. The mobile devices or their operators can control, request or influence the particular data content being delivered. The portable scanning device that includes a touch screen and a hermetically sealed body. The touch screen eliminates the need for a mechanical keyboard and therefore reduces the time required to clean or sanitize the scanner between uses. The portable scanning device also includes a removable memory device that is configured to store medical information. Because the portable scanning device does not include a hard drive, the cost and weight of the scanning device are reduced. Moreover, the portable scanning device includes a wireless USB that is configured to automatically transmit the medical information to a centrally located facility. The wireless USB facilitates reducing the amount of time required by the operator to download patient information thus increasing the operator's efficiency.

A technical effect of the various embodiments of the systems and methods described herein include at least one of automatically downloading patient data from a hand carried device to a central computer to reduce the amount of time required by an operator to download patient information.

While the invention has been described in terms of various specific embodiments, those skilled in the art will recognize that the invention can be practiced with modification within the spirit and scope of the claims.

What is claimed is:

1. A portable medical imaging device comprising:
a user interface configured to receive a user input
a display area on the user interface configured to display medical images
a processor module coupled to the user interface, the processor module configured to generate medical information and
a wireless communication interface configured to receive a wireless signal from a remote medical system and automatically transmit the medical information to the remote medical system in response to the received wireless signal.

2. A portable medical imaging device in accordance with claim 1 wherein the user interface comprises a touch screen.

3. A portable medical imaging device in accordance with claim 1 wherein the wireless communication interface comprises at least one of an wireless USB device, an Ethernet device, and a broadband device.

4. A portable medical imaging device in accordance with claim 1 wherein the remote medical system comprises a wireless universal service bus (WUSB) hub.

5. A portable medical imaging device in accordance with claim 1 further comprising a removable data storage device, said portable medical imaging device is configured to generate ultrasound information and store the ultrasound information directly to the removable data storage device.

6. A portable medical imaging device in accordance with claim 1 further comprising a removable flash-based random access memory (RAM) device, said processor is configured to generate ultrasound information and store the ultrasound information directly to the removable RAM device.

7. A portable medical imaging device in accordance with claim 1 further comprising a removable data storage device and an operating system installed on the removable data storage device.

8. A portable medical imaging device in accordance with claim 1 wherein the display the user interface, and the processor module are each located in a single hermetically sealed unit.

9. A medical imaging system comprising:
a data storage device;
a transmitter/receiver coupled to the data storage device; and
a portable medical imaging device having medical information stored thereon, the portable medical imagine device is configured to receive a wireless signal from transmitter/receiver and automatically transmit the medical information to the data storage device in response to the received signal.

10. A medical imaging system in accordance with claim 9 wherein the portable medical imaging device comprises:
a touch screen and
a display area programmed on the touchscreen.

11. A medical imaging system in accordance with claim 9 wherein the transmitter/receiver comprises a wireless universal service (WUSB) hub and the portable medical imaging device comprises a wireless communication interface, the wireless communication interface is configured to automatically transmit the medical information to the WUSB hub in response a signal received from the WUSB hub.

12. A medical imaging system in accordance with claim 9 wherein the portable medical imaging device further comprises a removable data storage device, said portable medical imaging device is configured to generate ultrasound information and store the ultrasound information directly to the removable data storage device.

13. A medical imaging system in accordance with claim 9 wherein the portable medical imaging device further comprises a removable flash-based random access memory (RAM) device, said portable medical imaging device is configured to generate ultrasound information and store the ultrasound information directly to the removable RAM device.

14. A medical imaging system in accordance with claim 9 wherein the portable medical imaging device further comprises a removable data storage device and an operating system that is installed on the removable data storage device.

15. A medical imaging system in accordance with claim 9 wherein the portable medical imaging device further comprises a body and a processor module hermetically sealed in the body.

16. A method for downloading medical information, said method comprising:
receiving at a portable medical imaging device a radio-frequency (RF) signal and downloading medical information from the portable medical imaging device to a remote medical system in response to the received RF signal.

17. A method in accordance with claim 16 further comprising:
generating the medical information and
storing the medical information directly to a removable memory device.

18. A method in accordance with claim 16 further comprising:
acquiring medical information using an ultrasound probe;
storing the medical information directly to a removable memory device; and
downloading the medical information from the removable memory device to the remote medical system in response to the received RF signal.

19. A method in accordance with claim 16 wherein the portable medical imaging device comprises a wireless communication interface and the remote medical system comprises a wireless USB hub, said method further comprising:
receiving at the wireless communication interface a radio-frequency (RF) signal transmitted from the wireless USB hub; and
downloading medical information from the portable medical imaging device to the remote medical system in response to the received RF signal.

20. A method in accordance with claim 16 further comprising automatically downloading medical information from the portable medical imaging device to a remote medical system in response to the received RF signal.
